SEQUENCE LISTING (14 sequences)

SEQ ID NO: 1 - DNA, 24 bp, Artificial
  Forward Primer for CAA gene
cttctaagatgccttctctctcca

SEQ ID NO: 2 - DNA, 24 bp, Artificial
  Reverse Primer for CAA gene
tattagaagcacaaacaaattgca

SEQ ID NO: 3 - DNA, 20 bp, Artificial
  Forward Primer for SKAA gene
cgagaccacctacaacagca

SEQ ID NO: 4 - DNA, 20 bp, Artificial
  Reverse Primer for SKAA gene
gcggtgatctctttctgcat

SEQ ID NO: 5 - DNA, 20 bp, Artificial
  Forward Primer for Trop C gene
ctacaaggctgcggtagagc

SEQ ID NO: 6 - DNA, 20 bp, Artificial
  Reverse Primer for Trop C gene
cagcacgaagatgtcgaagg

SEQ ID NO: 7 - DNA, 20 bp, Artificial
  Forward Primer for Trop T gene
atccccgatggagagagagt

SEQ ID NO: 8 - DNA, 20 bp, Artificial
  Reverse Primer for Trop T gene
acgagctcctcctcctcttt

SEQ ID NO: 9 - DNA, 19 bp, Artificial
  Forward Primer for Nkx2.5 gene
gattccgcagagcaactcg

SEQ ID NO: 10 - DNA, 21 bp, Artificial
  Reverse Primer for Nkx2.5 gene
ggagctgttgaggtgggatcg

SEQ ID NO: 11 - DNA, 20 bp, Artificial
  Forward Primer for GATA4 gene
tccaaaccagaaaacggaag

SEQ ID NO: 12 - DNA, 20 bp, Artificial
  Reverse Primer for GATA4 gene
aaggctctcactgcctgaag

SEQ ID NO: 13 - DNA, 20 bp, Artificial
  Forward Primer for BA gene
tccctggagaagagctacga

SEQ ID NO: 14 - DNA, 20 bp, Artificial
  Reverse Primer for BA gene
agcactgtgttggcgtacag

(19) United States
(12) Patent Application Publication
(10) Pub. No.: US 2014/0336074 A1
(43) Pub. Date: Nov. 13, 2014

(54) METHOD FOR PREPARING IMPROVED CARDIOMYCOCYTE-LIKE CELLS

(71) Applicant: SINGAPORE HEALTH SERVICES PTE LTD, Singapore (SG)

(72) Inventors: Se Ngie Winston Shim, Singapore (SG); Jean Ai Pearly Yong, Singapore (SG); En Hou Philip Wong, Singapore (SG)

(21) Appl. No.: 14/363,186

(22) PCT Filed: Dec. 6, 2012
(86) PCT No.: PCT/SG2012/000457

§ 371 Date: Jun. 5, 2014

(30) Foreign Application Priority Data
Dec. 6, 2011 (SG) 201109082-6

(51) Int. Cl. C12N 5/077; C12Q 1/68; G01N 33/50

(52) U.S. Cl. CPC C12N 5/0657; G01N 33/5061; C12Q 1/6881; G01N 33/5023; C12N 2501/585; C12N 2501/998; G01N 2500/10; C12Q 2600/136; C12Q 2600/158
USPC 506/9; 435/377; 435/29; 435/6.12; 435/7.92

(57) ABSTRACT

The present invention relates to the field of cell differentiation. In particular, the present invention relates to a method for preparing improved cardiomyocyte-like cells differentiated from at least one multipotent or pluripotent cell, comprising a step of contacting the cardiomyocyte-like cell(s) with at least one agent to promote and/or induce integrin subunit alpha-V (integrin αᵥ) activity, thereby modulating expression of at least one cardiac gene of said cardiomyocyte-like cells. It also relates to a method for screening for an agent capable of improving cardiomyocytes differentiated from at least one multipotent or pluripotent cell.

### FIELD OF THE INVENTION

The present invention relates to the field of cell differentiation. In particular, the present invention relates to a method for preparing improved cardiomyocyte-like cells differentiated from at least one multipotent or pluripotent cell. It also relates to a method for screening for an agent capable of improving cardiomyocytes differentiated from at least one multipotent or pluripotent cell.

### BACKGROUND OF THE INVENTION

Myocardial infarction (MI) results in necrosis, inflammation and scar formation in the myocardium. Such pathological insults place increasing mechanical demands on surviving cardiomyocytes (Boudoulas & Hatzopoulos, 2009). As cardiomyocytes have limited regenerative potential, loss of functional healthy tissue and subsequent left ventricular (LV) remodelling, eventually leads to pathological hypertrophic cardiomyopathy. Hypertrophy of the LV has been documented as a chronic response to MI and invariably progresses to heart failure (Hannigan et al., 2007). Chronic heart failure is a major health problem with patients experiencing debilitating quality of life.

Cardiac remodeling after MI is characterized by progressive and pathological interstitial fibrosis. During acute phase of cardiac repair, degradation of myocardial extracellular matrix (ECM) coupled with an influx of inflammatory cells and cytokines permits deposition of granulation tissue in the infarct region. At the site of tissue injury, granulation tissue composes of macrophages, myofibroblasts and neovascularisation. Activated myofibroblasts synthesize collagen and other ECM proteins to form dense scar tissue in the infarct in response to inflammatory mediators such as angiotensin II (Ang II) and transforming growth factor-β1 (TGF-β1). Macrophages drive the production of TGF-β1, an essential growth factor for fibroblast production, collagen synthesis and inhibition of collagen degradation (O'Kane & Ferguson, 1997; Sun & Weber, 2000). At the site of MI, increased expression adhesion molecules (inter-cellular adhesion molecule-1, ICAM-1) and chemoattractant cytokines (monocyte chemotactic protein-1, MCP-1) facilitate migration of inflammatory cells (e.g. macrophages) enabling scavenging of necrotic tissues (Lu et al., 2004). This couples with elevated expression of matrix metalloproteinase-1 (MMP-1) to result in remodeling of myocardial ECM by degradation of existing collagen I and III in the injured myocardium (Lu et al., 2004). MMP-9 has also been implicated in tissue remodelling and is thought to participate by cleaving collagen V at the amino-terminus (Niyibizi et al., 1994). Consequently, this process compromises structural integrity of the ventricles, resulting in myocyte slippage, wall thinning and rupture (Cleutjens et al., 1995b). Derangements in cardiomyocyte-ECM interactions cause the loss of cellular tensegrity and initiates anoikis in neighbouring healthy tissue (Michel, 2003). It is now well recognized that structural changes in the myocardial ECM can alter collagen-integrin-cytoskeletal-myofibril relations, thus affecting overall geometry and function of the heart (Spinale, 2007).

In non-cartilaginous tissues like the heart, collagen I, III and V are the predominant subtypes of the ECM (Breuls et al., 2009; Linehan et al., 2001). Collagen I is primarily a structural element of the myocardial ECM while collagen V represents a minor, but important component sequestered within collagen I fibres. However, collagen V levels increased in inflammation and scar tissue. The relative resistance of collagen V to mammalian collagenases makes it transiently available during tissue remodeling. The temporal availability of collagen V during active extracellular remodeling implies that it may play an important role in ECM remodeling and tissue stiffness (Breuls et al., 2009; Ruggiero et al., 1994). In fact, collagen V plays a deterministic role in collagenous fibril structure, matrix organization and stiffness (Fichard et al., 1995).

Binding of ECM to integrins provides a linkage between the ECM and cellular cytoskeleton. Integrins are heterodimeric receptors composed of non-covalently bound α and β subunits. (Brancaccio et al., 2006). Dynamic integrin-ECM interactions results in bidirectional signalling and determines cell morphology, gene expression, migration, proliferation, differentiation and death. Perkins et al. (2010) showed that integrin-mediated adhesion is mandatory for maintenance of the sarcomeric architecture. They proposed that disintegration of the Z-line and progressive muscle degeneration can occur once the adhesion complex comprising of integrins, talin or integrin linked kinase (ILK) is not replenished. In the myocardium, integrins can function as mechanotransducers that transmit mechanical ECM cues to the myocyte, resulting in changes to myocyte biology and function (Ross & Borg, 2001). Integrins α₂β₁, α₁β₁, α₃β₁, αᵥβ₃, α_{IIb}β₃ are collagen binding heterodimers and adhesion to collagen V has been reported to be primarily mediated by integrin α₂β₁ and α₁β₁ (Ruggiero et al., 1994). Integrins α₂β₁ and α₁β₁ may thus play a significant role in remodeling of the heart where there is increased collagen synthesis and collagen V expression, although other more recent reports have also shown that αᵥβ₃, not α₂β₁, may be important in cardiac differentiation of human mesenchymal stem cells (hMSCs) especially in conjunction with collagen V ECM; but αᵥβ₃, and in particular the αᵥ subunit has a minimal role in CLC adhesion to collagen V (Tan et al., 2010).

Increased ejection fraction (EF) and fractional shortening (FS) parameters, coupled with a reduction in the amount of fibrotic scar tissue have been highlighted following cellular therapy (Chacko et al., 2009). A previous study showed that cardiomyocyte-like cells (CLCs) that were differentiated from MSCs, improved systolic performance without compromising end-diastolic pressure of the infarcted myocardium when compared to MSCs. CLCs may facilitate hemodynamic recovery by preserving tissue elasticity in the collagen V-expressing peri-infarct borders. This unique cell/matrix relationship may be more conducive to a functionally adaptive remodeling response in maintaining contractile efficiency of post-infarcted myocardium (Tan et al., 2010).

Experimental data show that MSC transplantation inhibits LV remodelling and improves heart function in animals with MI (Xu et al., 2005). However, despite the ability of angiogenic mechanisms to reduce infarct mass, only partial restoration of ventricular contraction occurs as myocytes are not regenerated (Gaudette & Cohen, 2006). In addition, cardiac differentiation and retention of surviving transplanted MSCs in-vivo is limited (Feygin et al., 2007). Present methods of cardiac cell therapy may thus provide only modest benefits, likely due to low engraftment of transplanted cells in the infarcted myocardium.

There is thus an urgent need to improve methods of cardiac cell therapy. In particular, there is a need to improve the differentiation, engraftment, myocardial distribution and survival of cells used for cardiac cell therapy, replacement and/or transplantation. Ultimately, such improvement would allow greater recovery of mechanical function to the heart using cardiac cell therapy.

### SUMMARY OF THE INVENTION

According to a first aspect, the present invention provides a method for preparing improved cardiomyocyte-like cells comprising the steps of:
(i) differentiating at least one multipotent or pluripotent cell into cardiomyocyte-like cell(s); and
(ii) contacting the cardiomyocyte-like cell(s) with at least one agent to promote and/or induce integrin subunit alpha-V (integrin α_{V}) activity, thereby modulating expression of at least one cardiac gene of said cardiomyocyte-like cells.

According to another aspect, the present invention provides a method for screening for an agent capable of improving cardiomyocytes differentiated from at least one multipotent or pluripotent cell comprising screening for a candidate agent capable of promoting and/or inducing integrin subunit alpha-V activity. For example, this method may comprise the steps of:
(i) providing cardiomyocyte-like cell(s) differentiated from at least one multipotent or pluripotent cell;
(ii) contacting the cardiomyocyte-like cell(s) with the candidate agent; and
(iii) (a) screening for a candidate agent capable of modulating the expression of at least one cardiac gene when compared to the expression of said cardiac gene in cardiomyocyte-like cells absent the candidate agent; and/or
   (b) determining if the candidate agent promotes and/or induces integrin subunit alpha-V activity.

### BRIEF DESCRIPTION OF THE FIGURES

FIG. 1 shows the different collagen distribution pattern in (A) an intact myocardium and (B) an infarcted rat myocardium. FIG. 1(Ai) shows two higher magnification images of the boxed area (i) of FIG. 1(A) showing collagen I and III distribution in the perimysium while collagen V was expressed in the endomysial space. FIG. 1(Bii) shows two higher magnification images of the myocardium, epicardium and pericardium at the boxed area “ii” of FIG. 1(B). Collagen V was predominantly expressed at the peri-infarct border surrounding viable myocytes and vasculature structures in the infarct region. Collagen I and III were positively stained in the infarcted epicardium and pericardium. FIG. 1(Biii) shows two higher magnification images of the boxed area “ii” of FIG. 1(B) demonstrating severe thinning of the LV anterior wall. Co-localisation of collagen I and III extended from the pericardium into the infarcted myocardium whereas collagen I was primarily localised in the endocardium. Collagen V was expressed in the vessels and was sparsely distributed in the infarct. Scale bar: 200 μm.

FIG. 2 comprises six images showing how CLCs integrate into collagen V-rich cardiac syncytium. FIG. 2(A) shows that transplanted CLCs preferentially home to the collagen V-rich myocardial ECM. FIG. 2(B) shows that transplanted MSCs localised in the collagen I enriched infarct zone away from the collagen V peri-infarct region. FIG. 2(C) shows engraftment of CLCs in the α-actinin stained myocardium. FIG. 2(D) shows engrafted CLCs in the α-actinin stained myocardium showing an affinity towards collagen V matrix in the absence of collagen I staining. FIG. 2(E) shows that MSCs were embedded in the collagen I-rich infarct zone and were isolated from α-actinin expressing cardiomyocytes. FIG. 2(F) shows that collagen V was sparsely distributed in the infarcted region, but mainly surrounded viable myocytes at the peri-infarct border. Scale bar: 20 μm. MSCs: Mesenchymal stem cells; CLCs: Cardiomyocyte-like cells; Col I: Collagen I; Col V: Collagen V.

### Definitions

As used herein, the term “cardiomyocyte-like cells” is intended to mean cells sharing features with cardiomyocytes. Cardiomyocyte-like cells (CLCs) are further defined by morphological characteristics as well as by specific marker characteristics. However, for the purposes of this specification “improving” a CLC or a cardiomyocyte may mean causing any CLC or cardiomyocyte to improve in generative potential, engraftment, myocardial distribution, survival; for use in cardiac cell therapy, replacement and/or transplantation, and includes causing any CLC or cardiomyocyte, through the method of the present invention, to show an improvement in expression for any one of the cardiac genes compared to a CLC or cardiomyocyte prepared without the use of the present invention. For example, this improvement may be reflected by modulation of the expression at least one of certain cardiac genes known to be beneficial for differentiation of multipotent or pluripotent cells into CLCs or for enhancing recovery of cardiomyocytes to permit normal heart function after myocardial infarction. For example these genes may include one or more of Nkx2.5, GATA4, cardiac α-actin (CAA), skeletal muscle α-actin (SKAA), troponin T (Trop T), and/or troponin C (Trop C), titin, myosin light chain, myosin heavy chain, alpha actinin, tropomyosin. The modulation may cause the expression profile of the at least one such cardiac gene to more closely resemble the expression profile of such genes in healthy cardiomyocytes. The modulation may be an increased expression of at least one such cardiac gene.

As used herein, the term “pluripotent” refers to the potential of a stem cell to make any differentiated cell of an organism. Pluripotent stem cells can give rise to any fetal or adult cell type. However, alone they cannot develop into a fetal or adult organism because they lack the potential to contribute to extraembryonic tissue, such as the placenta.

As used herein, the term “multipotent” refers to the potential of a stem cell to give rise to a subset of cell lineages, for example within a particular tissue, organ or physiological system.

“Mesenchymal stem cells” (MSCs) refer to multipotent stem cells that can differentiate into a variety of cell types including: osteoblasts (bone cells), chondrocytes (cartilage cells) and adipocytes (fat cells).

The term “mesenchymal stem cell-like cells” is intended to mean cells sharing features with mesenchymal stem cells. For example, mesenchymal stem cell-like cells share growth characteristics, biochemical activity and markers resembling that of mesenchymal stem cells.

“Modulating” refers to altering a biological process. For example, modulating gene expression may refer to increasing, decreasing or otherwise changing the expression of a given gene. Each of the various steps in gene expression may be modulated, such as transcriptional initiation, RNA processing, and post-translational modification of a protein. Modulation may be by any suitable agent, for example a repressor agent may impede RNA polymerase and thus impede expression of a gene. An inhibitory antibody may bind to and impede function of an integrin subunit such as integrin subunit alpha-1, possibly preventing or reducing a cascading pathway of interactions that eventually exert a modulating effect on the expression of one or more cardiac genes.

Conversely, an activating antibody may bind to and activate an integrin subunit such as integrin subunit alpha-V, possibly increasing the cascade of interactions that eventually exert a modulating effect on the expression of one or more cardiac genes and enhancing differentiation of the multipotent or pluripotent cells into CLCs.

“Promoting” the activity of a protein may refer to allowing the protein to function normally or enhancing its normal function. For example, in the context of CLC transplantation into a patient with myocardial infarction as part of cardiac cell therapy, promoting the activity of an integrin protein may comprise increasing its ability to encourage differentiation of multipotent or pluripotent cells into CLCs that more closely resemble healthy cardiomyocytes and may further include encouraging integrin-ECM signalling to allow recovery of normal heart function.

“Short inhibitory RNA” refers to a class of short, for example about 20-nucleotide long double-stranded RNA molecules which are used in the technique of RNA interference (RNAi) to inhibit expression of specific genes. They are also known as small interfering RNA or short interfering RNA. “MicroRNA” (miRNA) refers to a class of short RNA molecules, for example about 22 nucleotides long, which may be used to obtain a similar inhibitory effect. miRNAs are post-transcriptional regulators that bind to complementary sequences on target messenger RNA transcripts (mRNAs), usually resulting in translational repression or target degradation and gene silencing. miRNAs may occur naturally.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “dominant negative”, as used herein and unless otherwise indicated, may refer to a gene product arising from a dominant negative gene. It generally refers to a mutant gene product such as a protein, which is able to disrupt the activity of its wild-type counterpart. For example, a dominant negative protein may still interact with the same elements as the wild-type product, but may not support the cascade of additional interactions which are supported in its wild-type counterpart. A protein that is functional as a dimer, such as an integrin, may have a dominant negative form that lacks the functional domain but retains the dimerization domain, causing a dominant negative phenotype because some fraction of protein dimers would be missing one of the functional domains.

Conversely, the term “dominant active” may refer to a gene product arising from a dominant positive gene. It generally refers to a mutant gene product such as a protein, which is able to increase or encourage the activity of its wild-type counterpart. For example, a dominant active protein may support the cascade of interactions which are supported in its wild-type counterpart but may require less or no interaction with activating elements that are required to activate the wild-type product. A protein that is functional as a dimer, such as an integrin, may have a dominant active form that has a normal functional domain but a dimerization domain that dimerizes more easily than the wild-type or that does not dissociate easily after dimerization, causing a dominant active phenotype because the fraction of dimerized protein would be greater than that observed in the wild-type protein. The dominant active gene product may be constitutively active. “Constitutively active” with respect to a polypeptide or protein means that the polypeptide or protein is functionally active independent of pathway activation and/or stimulation, such as phosphorylation.

“RGD peptides” as used in this specification refer to peptides with at least one arginine-glycineaspartate (RGD) sequence which can mimic cell adhesion proteins and bind to integrins. The RGD sequence is the cell attachment site of a large number of adhesive extracellular matrix, blood, and cell surface proteins, and nearly half of the over 20 known integrins recognize this sequence in their adhesion protein ligands. Some other integrins bind to related sequences in their ligands. The integrin-binding activity of adhesion proteins can be reproduced by short synthetic peptides containing the RGD sequence. Such peptides promote cell adhesion when insolubilized onto a surface, and inhibit it when presented to cells in solution. Reagents that bind selectively to only one or a few of the RGD-directed integrins can be designed by cyclizing peptides with selected sequences around the RGD and by synthesizing RGD mimics. This may have an inhibitory effect by reducing the activity of integrins. Conversely, a reagent comprising one or more RGD sequences may promote activity of an integrin, for example by arranging the RGD sequences to increase the local concentration of the integrin at the desired site. The RGD sequence may be partially or completely exposed so as to adjust binding efficacy to integrins.

“Dimer” as used in this specification includes both heterodimers assembled from or comprising different subunits, and homodimers assembled from or comprising identical subunits.

### DETAILED DESCRIPTION OF THE INVENTION

To enable improved methods of cardiac cell therapy, in a first aspect the present invention provides a method for preparing improved cardiomyocyte-like cells (CLCs) comprising the steps of:
(i) differentiating at least one multipotent or pluripotent cell into cardiomyocyte-like cell(s); and
(ii) contacting the cardiomyocyte-like cell(s) with at least one agent to promote and/or induce integrin subunit alpha-V (integrin α_{V}) activity, thereby modulating expression of at least one cardiac gene of said cardiomyocyte-like cells.

The at least one agent(s) used in the method may promote and/or induce integrin subunit alpha-V activity by itself or as part of a dimer. Any suitable agent may be used to produce this effect, for example the at least one agent(s) may comprise an activating antibody, a RGD peptide and/or a constitutively active form of alpha-V integrin subunit.

Promoting and/or inducing the activity of integrin subunit alpha-V may comprise promoting and/or inducing integrin subunit alpha-V gene expression. This may be at the transcriptional and/or translational levels.

The method for preparing improved CLCs may further comprise contacting the multipotent, pluripotent and/or cardiomyocyte-like cell(s) with collagen prior to or concomitant with the at least one agent. The collagen may comprise collagen IV, V and/or XI. The collagen may comprise collagen V.

For example, step (ii) of the method for preparing improved CLCs may comprise modulating cardiac gene expression at the transcriptional and/or translational level.

The method may comprise enhancing the expression of at least one cardiac gene. The at least one cardiac gene may comprise GATA4, Nkx2.5, cardiac α-actin, cardiac troponin T, and/or cardiac troponin C. The at least one cardiac gene may comprise cardiac α-actin, cardiac troponin T, and/or cardiac troponin C. The at least one cardiac gene may comprise cardiac troponin T, and/or cardiac troponin C.

The method may comprise a step of modulating the expression of at least one cardiac gene of the cardiomyocyte-like cells. Examples of cardiac genes may include sarcomeric α-actin, sarcomeric α-actinin, desmin, skeletal/cardiac specific titin, sarcomeric α-tropomyosin, cardiac troponin I, sarcomeric MHC, SERCA2 ATPase, connexin-43, GATA binding protein 4 (GATA4), Nkx2.5, myocyte enhancing factor 2A, myocyte enhancing factor 2C, myocyte enhancing factor 2D, cardiac α-actin, skeletal muscle α-actin, cardiac troponin T, cardiac troponin C, and L-type calcium α1c.

The CLCs prepared in the present method may be suitable and/or for use in transplantation. For example, they may be suitable for use in transplantation into a patient with myocardial infarction as part of cardiac cell therapy.

The invention includes a method for screening for an agent capable of improving cardiomyocytes. According to another aspect the present invention provides a method for screening for an agent capable of improving cardiomyocytes differentiated from at least one multipotent or pluripotent cell comprising screening for a candidate agent capable of increasing the integrin subunit alpha-V activity. This method may comprise the steps of:
(i) providing cardiomyocyte-like cell(s) differentiated from at least one multipotent or pluripotent cell;
(ii) contacting the cardiomyocyte-like cell(s) with the candidate agent; and
(iii) (a) screening for a candidate agent capable of modulating the expression of at least one cardiac gene when compared to the expression of said cardiac gene in cardiomyocyte-like cells absent the candidate agent; and/or
   (b) determining if the candidate agent promotes and/or induces integrin subunit alpha-V activity.

The ability of the candidate agent to modulate the expression of the at least one cardiac gene and/or to promote and/or induce integrin subunit alpha-V activity may be indicative of its ability to improve cardiomyocytes differentiated from at least one multipotent or pluripotent cell.

The screening method may comprise detecting for enhanced expression of at least one cardiac gene in cardiomyocyte-like cell(s) differentiated from at least one multipotent or pluripotent cell contacted with the candidate agent. For example, the enhanced expression of GATA4, Nkx2.5, cardiac α-actin, cardiac troponin T, and/or cardiac troponin C genes in cardiomyocyte-like cells differentiated from at least one multipotent or pluripotent cell contacted with the candidate agent may be detected. The enhanced expression of cardiac α-actin, cardiac troponin T, and/or cardiac troponin C genes in cardiomyocyte-like cells differentiated from at least one multipotent or pluripotent cell contacted with the candidate agent may be detected. The enhanced expression of cardiac troponin T, and/or cardiac troponin C genes in cardiomyocyte-like cells differentiated from at least one multipotent or pluripotent cell contacted with the candidate agent may be detected.

According to a particular embodiment, the screening method may also comprise detecting and comparing the expression of at least one cardiac gene in cardiomyocyte-like cells contacted with and absent the candidate agent.

For the present screening method, reference to modulation of gene expression of at least one cardiac gene or enhanced expression of selected cardiac genes may be at the transcription and/or translation level. For example, this method may comprise detecting for the transcription and/or translation product. Detection in this method may comprise using real-time PCR, microarray analysis, ELISA and/or immunoblotting.

This method for screening for an agent capable of improving cardiomyocytes, may be performed in vitro.

The present invention is also described in the following statements:
1. A method for preparing improved cardiomyocyte-like cells comprising the steps of:
   (i) differentiating at least one multipotent or pluripotent cell into
   (ii) contacting the cardiomyocyte-like cell(s) with at least one agent to reduce and/or inhibit integrin subunit alpha-1 (integrin α₁) activity, thereby modulating expression of at least one cardiac gene of said cardiomyocyte-like cells.
2. The method according to statement 1, wherein the agent reduces and/or inhibits integrin subunit alpha-1 activity by itself or as part of a dimer.
3. The method according to statement 1 or 2, wherein the agent comprises an antibody, a RGD peptide and/or a dominant negative form of alpha-1 integrin subunit.
4. The method according to statement 1, wherein reducing and/or inhibiting the activity of integrin subunit alpha-1 comprises reducing and/or inhibiting integrin subunit alpha-1 gene expression.
5. The method according to statement 4, reducing and/or inhibiting the activity of integrin subunit alpha-1 comprises reducing and/or inhibiting integrin subunit alpha-1 gene expression at the transcription and/or translational levels.
6. The method according to statement 4 or 5, wherein the agent comprises short inhibitory RNA and/or microRNA.
7. The method according to any one of the preceding statements, further comprising contacting the multipotent, pluripotent and/or cardiomyocyte-like cell(s) with collagen prior to or concomitant with the agent.
8. The method according to statement 7, wherein the collagen comprises collagen IV, V and/or XI.
9. The method according to statement 7 or 8, wherein the collagen comprises collagen V.
10. The method according to any one of the preceding statements, wherein step (ii) comprises modulating cardiac gene expression at the transcriptional and/or translational level.
11. The method according to any one of the preceding statements, wherein said cardiac gene comprises sarcomeric α-actin, sarcomeric α-actinin, desmin, skeletal/cardiac specific titin, sarcomeric α-tropomyosin, cardiac troponin I, sarcomeric MHC, SERCA2 ATPase, connexin-43, GATA binding protein 4, Nkx2.5, myocyte enhancing factor 2A, myocyte enhancing factor 2C, myocyte enhancing factor 2D, cardiac α-actin, skeletal muscle α-actin, cardiac troponin T, cardiac troponin C, and L-type calcium α1c.
12. The method according to any one of statements 1 to 10, wherein step (ii) comprises enhancing the expression of at least one cardiac gene.
13. The method according to statement 12, wherein the at least one cardiac genes comprises cardiac α-actin, cardiac troponin T, and/or cardiac troponin C.
14. The method according to any one of the preceding statements, wherein the cardiomyocyte-like cells are suitable and/or for use in transplantation.
15. A method for screening for an agent capable of improving cardiomyocytes differentiated from at least one multipotent or pluripotent cell comprising screening for a candidate agent capable of reducing the integrin subunit alpha-1 activity.
16. The method according to statement 15, comprising the steps of:
   (i) providing cardiomyocyte-like cell(s) differentiated from at least one multipotent or pluripotent cell;
   (ii) contacting the cardiomyocyte-like cell(s) with the candidate agent; and
      (iii) (a) screening for a candidate agent capable of modulating the expression of at least one cardiac gene when compared to the expression of said cardiac gene in cardiomyocyte-like cells absent the candidate agent; and/or (b) determining if the candidate agent reduces and/or inhibits integrin subunit alpha-1 activity.
17. The method according to statement 15 or 16, comprising detecting for enhanced expression of selected cardiac genes in cardiomyocyte-like cell(s) differentiated from at least one multipotent or pluripotent cell contacted with the candidate agent.
18. The method according to any one of statements 15 to 17, comprising detecting for enhanced expression of cardiac α-actin, cardiac troponin T, and/or cardiac troponin C genes in cardiomyocyte-like cells differentiated from at least one multipotent or pluripotent cell contacted with the candidate agent.
19. The method according to any one of statements 15 to 18, comprising detecting and comparing the expression of at least one cardiac gene in cardiomyocyte-like cells contacted with and absent the candidate agent.
20. The method according to any one of statements 16 to 19, wherein modulating or enhanced cardiac gene expression is at the transcription and/or translation level.
21. The method according to any one of statements 16 to 20, comprising detecting for the transcription and/or translation product.
22. The method according to statement 21, wherein detection comprises using real-time PCR, microarray analysis, ELISA and/or immunoblotting.
23. The method according to any one of the preceding statements, wherein the method is an in vitro method.

Having now generally described the invention, the same will be more readily understood through reference to the following examples which are provided by way of illustration, and are not intended to be limiting of the present invention.

### EXAMPLES

### Materials and Methods

Isolation and Culture of Bone Marrow Derived MSCs

Bone marrow was isolated from the sternum of patients undergoing open-heart surgery. They were collected in 17 IU/ml heparin using a 23-gauge needle. Bone marrow aspirates were topped up to 15 ml with Dulbecco's modified Eagle's medium-low glucose (DMEM-LG, GIBCO) supplemented with 10% fetal bovine serum (FBS, Hyclone) and 1% penicillin-streptomycin (Gibco, Invitrogen). To deplete bone marrow asiprates of mature blood lineages, 15 ml of bone marrow blood mixture was overlaid onto 15 ml of Histopaque®⁻077 (Sigma-Aldrich) and centrifuged for 1500 rpm (Kubota Centrifuge) for 30 minutes at 4° C. The enriched cell fraction was collected from the interphase, washed once with 5 ml of media and centrifuged at 1200 rpm (Kubota Centrifuge) for 10 minutes. Resuspended cells were then transferred into tissue culture flasks with basal normal growth medium (NGM) comprising (DMEM-LG) supplemented with 10% FBS for 9-11 days to yield plastic adherent MSCs. Subconfluent cells were harvested using 1× Trypsin-EDTA solution for endothelial cell cultured (Sigma-Aldrich) 14-21 days after initial plating and maintained as MSCs in basal NMG or differentiated towards CLCs in a myogenic differentiation medium (MDM) as previously described (Shim et al., 2004).

Type V collagen (Sigma-Aldrich) and Type I collagen (BD™) were coated on 6-well plates or tissue culture flasks at 10 μg/cm² for 3 hours at room temperature. Plates and flasks were washed twice with phosphate buffered saline (PBS) and kept at 4° C. until required.

Fluorescence Microscopy

Frozen tissue sections of the explanted ventricular rat hearts were fixed in 4% paraformaldehyde (PFA), permeabilised with 0.1% Triton X-100, and further blocked in 5% bovine serum albumin (BSA). This was followed by overnight incubation at 4° C. with primary antibodies, including collagen I (Southern Biotech), collagen III (Affinity Bioregent) collagen V (Biotrend) and anti-a-sarcomeric actinin (Sigma-Aldrich) diluted in 1% BSA. Sections were incubated with Alexa Fluor® 488/555/660—conjugated secondary antibodies (Molecular Probes) in 0.1% BSA at room temperature for 3 hours before staining the nuclei with DAPI. Immunofluorescence microscopy was performed with Zeiss Axiovert 200 M fluorescence microscope, using the Metamorph software (version 6.2, Molecular Devices) or Leica MZ 16 FA Fluorescence Steromicroscope, using the Leica Application Suite software (Version 3.3.0, Leica).

Flow Cytometry

Sternum-derived bone marrow MSCs were differentiated into CLCs and characterized by flow cytometry after 14 days in a MDM. CLCs cultured on uncoated, collagen I or V coated tissue culture flasks were stained with antibodies directed towards integrin subunits α₁ (Abcam), α₂ (Santa Cruz), αᵥ (Fitzgerald), β₁ (Chemicon), β₃ (Cell Signaling). Cells were treated with Fix & Perm® Cell Permeabilisation Kit (Invitrogen) and subsequently blocked in PBS containing 5% BSA, 1% FBS and 5 mM ethylenediaminetetracetic acid (EDTA) for 30 minutes at 4° C. on a roller. CLCs were then incubated with directly conjugated antibodies for 30 minutes at 4° C. Indirectly conjugated antibodies were incubated for 2 hours at 4° C. and subsequently stained with their respective Alexa Fluor® 555 conjugated secondary antibodies (Invitrogen) for 2 hours at 4° C. Isotype controls were stained in parallel with the test samples. Samples were washed in PBS containing 2% BSA, 2% FBS and 5 mM EDTA after each antibody staining and fixation step. All samples were fixed in PBS containing 4% PFA/PBS, washed and resuspended in PBS containing 2% FBS and 0.09% sodium azide (NaZ). Data analysis was performed using FACSDiva software (version 6.1.2, BD™) FlowJo software (version 6.4, Tree Star, Inc.). Histogram overlays were performed and the change in median fluorescence intensity and overton subtraction percentages were computed.

Integrin Neutralisation Assays

Integrin neutralisation assays were performed on CLCs using neutralising antibodies against the integrin α₁ (Millipore) subunit and αᵥβ₃ (Millipore) heterodimer, at 1 μg/ml and 10 μg/ml respectively. CLCs treated with 1 μg/ml or 10 μg/ml isotype IgG (Abcam/Dako) antibodies and untreated CLCs served as control. After trypsin disgestion, CLCs were incubated with neutralising and isotype control antibodies for 2 hours at 4° C. 50,000 untreated and treated CLCs were seeded on collagen V pre-coated 6-well plates. Plated CLCs were harvested after 72 hours of culture at 37° C., 5% CO₂. Total RNA was extracted using the RNeasy Mini Kit (Qiagen) and treated with RNAse free DNase solution (Qiagen). DNAse treated RNA samples were stored at −80° C. until required.

RNAi Transfection to Knockdown α_{V} and Alpha1 Integrins

Pre-validated RNAi molecules that specifically targeting α_{V} or α₁ integrin were purchased from Applied Biosystems (Life Technologies, USA). Human cardiomyocyte-like cells (CLCs) were cultured on collagen V-coated tissue culture 6-well plate with 5×10⁴ cells two days prior to transfection with individual RNAi molecule following manufacturer's instructions using MATra RNAi magnetic transfection system (Promokine, USA). The transfected CLCs were harvested at 48 h and RNA extracted for 1^{st} strand synthesis for cDNA using Superscript III (Life Technologies, USA) and subsequently used for real-time PCR to detect changes in the gene expression of targeted integrins and cardiac markers following manufacturer's instructions (Qiagen, USA). The relative gene expression levels were analyzed using CLCs transfected with sequence scrambled control RNAi molecule (Applied Biosystemts, Life Technologies) for normalization.

Real-Time Reverse Transcriptase Polymerase Chain Reaction for Quantitation of Cardiac Gene Expression

First strand cDNA was synthesized from total RNA using the SuperScript™ III First-Strand Synthesis System (Invitrogen) and equal concentrations of cDNA were loaded into tubes containing QuantiFast SYBR Green PCR mastermix (Qiagen). Real-time reverse transcriptase polymerase chain reaction (RT-PCR) was performed on the Rotor-Gene Q thermocycler (Qiagen) using standard cycling parameters and relative gene expression of the following cardiac transcripts was quantitated using the ΔΔC_{T} method. These transcripts include β actin (BA), cardiac α-actin (CAA), skeletal muscle α-actin (SKAA), troponin T (Trop T), troponin C (Trop C), Nkx2.5 and GATA4 (Sigma-Aldrich). Target gene expression values were normalised relative to the untreated CLCs. BA served as a housekeeping gene for the real time RT-PCR experiments. No template controls were concurrently processed with test samples to rule out the presence of contaminated reagents and nucleic acids.

TABLE 1
Primer sequences for real time reverse transcriptase polymerase chain
reaction (RT-PCR). Transcripts obtained from RT-PCR assays were all
less than 200 bp. CAA, cardiac α-actin; SKAA, skeletal muscle α-actin;
Trop T, troponin T; Trop C, troponin C; BA, β actin.

| | Acession | | Product |
|---|---|---|---|
| Gene | Number | Primer Sequence | Size (bp) |
| CAA | NM_005159 | 5′-CTTCTAAGATGCCTTCTCTCTCCA-3′ | 147 |
| | | 5′-TAT TAG AAG CAC AAA CAA ATT GCA-3′ | |
| SKAA | NM_001100.3 | 5′-CGAGACCACCTACAACAGCA-3′ | 132 |
| | | 5′-GCGGTGATCTCTTTCTGCAT-3′ | |
| Trop C | NM_003280.2 | 5′-CTACAAGGCTGCGGTAGAGC-3′ | 76 |
| | | 5′-CAGCACGAACATGTCGAAGG-3′ | |
| Trop T | NM_000364.2 | 5′-ATCCCCGATGGAGAGAGAGT-3′ | 128 |
| | | 5′-ACGAGCTCCTCCTCCTCTTT-3′ | |
| Nkx2.5 | NM_004387 | 5′-GATTCCGCAGAGCAACTCG-3′ | 105 |
| | | 5′GGAGCTGTTGAGGTGGGATCG-3′ | |
| GATA4 | NM_002052.3 | 5′-TCCAAACCAGAAAACGGAAG-3′ | 77 |
| | | 5′-AAGGCTCTCACTGCCTGAAG-3′ | |
| BA | NM_001101 | 5′-TCCCTGGAGAAGAGCTACGA-3′ | 194 |
| | | 5′-AGCACTGTGTTGCCGTACAG-3′ | |

Cell Labelling

CLCs were labeled with 1 mmol/L Vybrant CellTracker chloromethyldialkylcarbocyanie (CM-Dil; Molecular Probes) overnight at 37° C. and rinsed 3 times before trypsin disgestion and transplantation. MSCs were labeled with 10 mmol/L Vybrant carboxy fluorescein diacetate succinimidyl ester (CFDA-SE; Molecular Probes). Cells were resuspended in a final concentration of 1×10⁶/0.1 ml to 5×10⁶/0.2 ml.

Rat Myocardial Infarction Model

MI was created in n=20 female Wistar rats per group. Each rat weighs approximately 350-400 g in body weight. The animals were subjected to left thoracotomy and the left anterior descending artery (LAD) was exposed and ligated. After which rats were allowed a week for recovery before given treatment of either injection with labeled cells or placebo to the area of infarction. Cyclosporin A was administered at a dose of 5 mg/kg body weight at 3 days before and daily following treatment for 6 weeks until end point.

Echocardiography

Baseline echocardiography was performed on each rat before MI and 6 weeks after treatment. Echocardiography images were acquired using Vivid 7 ultrasound machine (General Electric VingMed) equipped with i13L linear probe operated at 14 MHz. Rats were anesthetised using 1%-2% isofluorane with 1 L/hr oxygen and then fixed in the supine position on a heated platform. Rats were then shaved at the chest and abdominal areas before electrocardiography (ECG) electrodes were placed onto the left and right leg as well as the left upper extremity. All analysis was performed offline with EchoPAC workstation (General Electric Healthcare).

Statistical Analysis

One-way analysis of variance (ANOVA) was used to determine statistical significance between different treatment groups. Tukey Honestly Significant Difference (HSD) post-hoc analyses were used to determine statistical significance between treatment groups using SPSS 13 software (SPSS Inc.). p<0.05 was considered statistically significant. All data are presented as mean±standard deviation (SD).

### Results

Integrin Expression and Cardiac Differentiation

Flow cytometric analysis showed that αᵥ and β₁ were the predominant subunits of integrins in CLCs, independent of substrate surface (Table 2). In comparison to collagen V matrix, CLCs cultured on collagen I showed a higher expression of integrin α₁ (59.4±13.7% vs. 78.0±0.9%) and β₃ (44.7±10.6% vs. 56.0±21.8%) subunits. Furthermore, with the exception of α₁ subunit, α₂, αᵥ, β₁ and β₃ integrins in CLCs cultured on either collagen matrices showed a reduction of expression in comparison to CLCs cultured on polystyrene tissue culture surface.

TABLE 2
Flow cytometric analysis showed that integrin αᵥ
and β₁ were the predominant subunits in CLCs.

| Integrin | CLC | | |
|---|---|---|---|
| Subunit | Uncoated | Collagen I | Collagen V |
| α1 | 58.5 ± 1.8 | 78.0 ± 0.9 | 59.4 ± 13.7 |
| α2 | 59.4 ± 6.1 | 39.9 ± 11.5 | 44.3 ± 12.8 |
| αv | 91.6 ± 0.5 | 83.8 ± 5.7 | 81.8 ± 4.6 |
| β1 | 93.4 ± 0.8 | 78.5 ± 8.6 | 79.1 ± 8.6 |
| β3 | 57.6 ± 2.4 | 56.0 ± 21.8 | 44.7 ± 10.6 |

CLCs cultured on collagen I showed increased levels of α₁ and β₃. Data were derived from 3 independent experiments and the overton percentage positive results are expressed as mean ± SD. MSCs, Mesenchymal stem cells; CLCs, Cardiomyocyte-like cells.

Enhanced Cardiac Gene Expression in CLCs Via Integrin α₁ and β₃ on Collagen V Matrices

A previous report showed that collagen V matrix enhanced cardiac gene expression when compared to CLCs seeded on collagen I matrix. Collagen V selectively upregulated expression of cardiac transcription factors (GATA4, Nkx2.5), calcium handling transporter (RyR2) and sarcomeric myofilament proteins (Trop T, Trop C, SKAA) in CLCs (Tan et al., 2010). Neutralisation of αᵥβ₃ integrin or α₁ subunit in this study did not affect CAA and SKAA gene expression in CLCs that were cultured on collagen V matrix (Table 3A). Furthermore, no significant changes in Nkx2.5 or GATA4 expression was observed in α₁ subunit neutralised CLCs. However, Nkx2.5 down regulation was observed in CLCs neutralised with αᵥβ₃, although similar down regulation was also evident in the isotype control experiment. Gene expression of Trop C reduced significantly after αᵥβ₃ integrin neutralisation. In contrary, α₁ subunit neutralisation upregulated Trop C expression. Furthermore, there was a concomitant upregulation of Trop T following α₁ integrin neutralisation.

αᵥ Integrin Promotes Cardiac Differentiation of CLCs

Consistent with α_{V}β₃ antibody neutralization results in Table 3A and a previous report (Tan et al. 2010), RNAi towards α_{V} integrin confirmed its key role in promoting cardiac differentiation of CLCs. There was a marked reduction in gene expression of targeted α_{V} integrin (but not α₁) with corresponding reduction in troponin C, troponin T, GATA4 and Nkx2.5 in the CLCs. In contrast, there was a minimal reduction in expression levels of troponin C and troponin T despite marked reduction of α₁ integrin (but not α_{V}) in the transfected CLCs with RNAi against α₁ integrin (Table 3B).

TABLE 3A
CLCs cultured on collagen V were treated with integrin α₁ (1 μg/ml) neutralising antibodies. Untreated and isotype IgG (1 μg/ml)
treated CLCs served as controls for this experiment. Optimal concentrations of test and control antibodies were predetermined in a series
of titration experiments. CLCs cultured on collagen V were treated with integrin αᵥβ₃ (10 μg/ml) neutralising antibodies. Untreated and
isotype IgG (10 μg/ml) treated CLCs served as controls for this experiment. Results are expressed as mean ± SD. CAA, cardiac α-actin;
SKAA, skeletal muscle α-actin; Trop T, troponin T; Trop C, troponin C, N.D., not done; MSCs, Mesenchymal stem cells; CLCs,
Cardiomyocyte-like cells.

| Integrin | | | | | | | | | | | | |
|---|---|---|---|---|---|---|---|---|---|---|---|---|
| Neutralisation | CAA | | SKAA | | Trop C | | Nkx2.5 | | Trop T | | GATA4 | |
| Antibody | Isotype | Test | Isotype | Test | Isotype | Test | Isotype | Test | Isotype | Test | Isotype | Test |
| αᵥ β₃ | 0.87 ± | 1.00 ± 0.10 | 1.32 ± | 1.15 ± 0.00 | 0.94 ± | 0.12 ± 0.02 | 0.23 ± | 0.22 ± 0.16 | N.D. | N.D. | N.D. | N.D. |
| | 0.00 | | 0.13 | | 0.10 | | 0.21 | | | | | |
| α₁ | 0.52 ± | 0.91 ± 0.02 | 0.99 ± | 1.02 ± 0.10 | 1.17 ± | 1.45 ± 0.40 | 1.02 ± | 0.90 ± 0.22 | 1.04 ± | 1.63 ± | 0.87 ± | 0.63 ± |
| | 0.05 | | 0.12 | | 0.11 | | 0.22 | | 0.05 | 0.16 | 0.00 | 0.45 |

TABLE 3B
RNAi inhibition of integrins reduced cardiac gene expression. Data presented as mean ± standard deviation normalized against
sequence scrambled RNAi treated cells. Each experiment was performed in triplicates.

| | Gene Expression (normalized) | | | | | | | |
|---|---|---|---|---|---|---|---|---|
| RNAi | αᵥ integrin | α₁ integrin | CAA | SkAA | Trop C | Trop T | GATA4 | Nkx2.5 |
| αᵥ | 0.07 ± 0.04 | 1.97 ± 0.67 | 1.55 ± 1.12 | 1.08 ± 0.33 | 0.69 ± 0.29 | 0.48 ± 0.08 | 0.41 ± 0.20 | 0.27 ± 0.08 |
| integrin | | | | | | | | |
| α₁ | 1.34 ± 0.79 | 0.08 ± 0.05 | 1.28 ± 0.93 | 1.12 ± 0.34 | 0.83 ± 0.12 | 0.93 ± 0.71 | 0.55 ± 0.04 | 0.70 ± 0.46 |
| integrin | | | | | | | | |

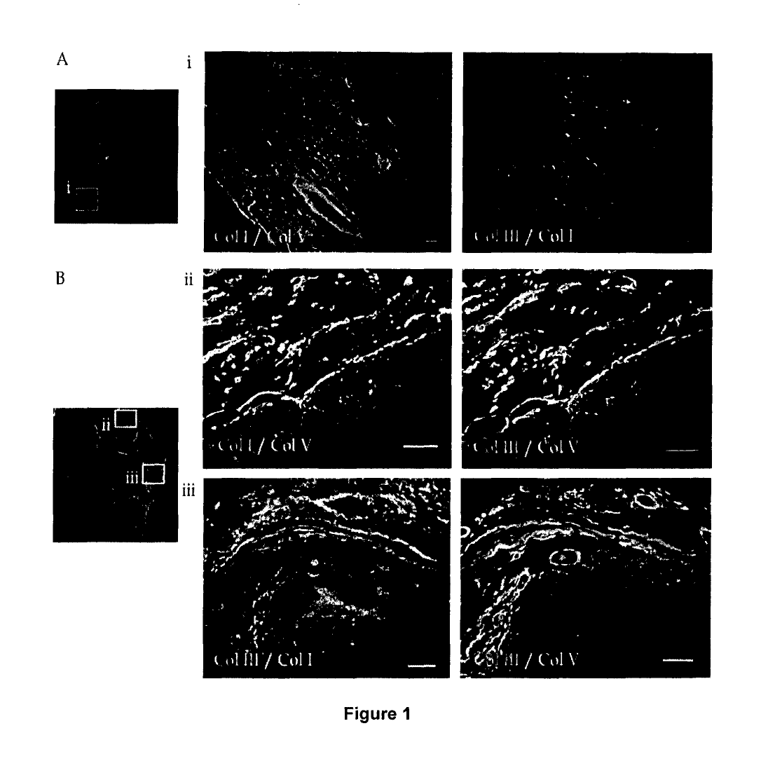

Consistent with a previous report (Tan et al., 2010), collagen I as the main constituent of cardiac ECM in intact rat myocardium was found to co-localise with collagen III matrix in the epicardium and perimysial space between major muscle bundles dispersed throughout the myocardium (FIG. 1A). On the other hand, collagen V was predominantly observed in the endomysial space surrounding healthy cardiomyocytes and in the perivascular structures within the myocardium. Following MI by ligating the left anterior descending artery, significant wall thinning was observed in the anterior wall of the LV (FIG. 1B). Accumulation of collagen matrices was evident in the infarcted and non-infarcted zones 7 weeks post infarction. Spatial remodeling and redistribution of collagen matrices were observed whereby perimysial collagen I diminished significantly and upregulation of collagen I and III was observed in the pericardium and epicardium of infarcted as well as non-infarcted zones. Furthermore, fibrosis consisted mainly of collagen I matrix was prominently found in the endocardium of infarcted zone while it co-localized with collagen III matrix in the pericardium/epicardium of the non-infarcted and infarcted zones (FIGS. 1Bii & 1Biii). In contrast, collagen V fibrils were sparsely detected in the epicardium of infarct, but were prominently found in the perivascular structures within the infarct (FIG. 1Biii). In contrast to redistribution of collagen I matrix, collagen V remained in the endomysial matrix of individual muscle fibres in the non-infarcted borders (FIG. 1Bii) and also surrounding isolated, but viable cardiac fibres in the infarct.

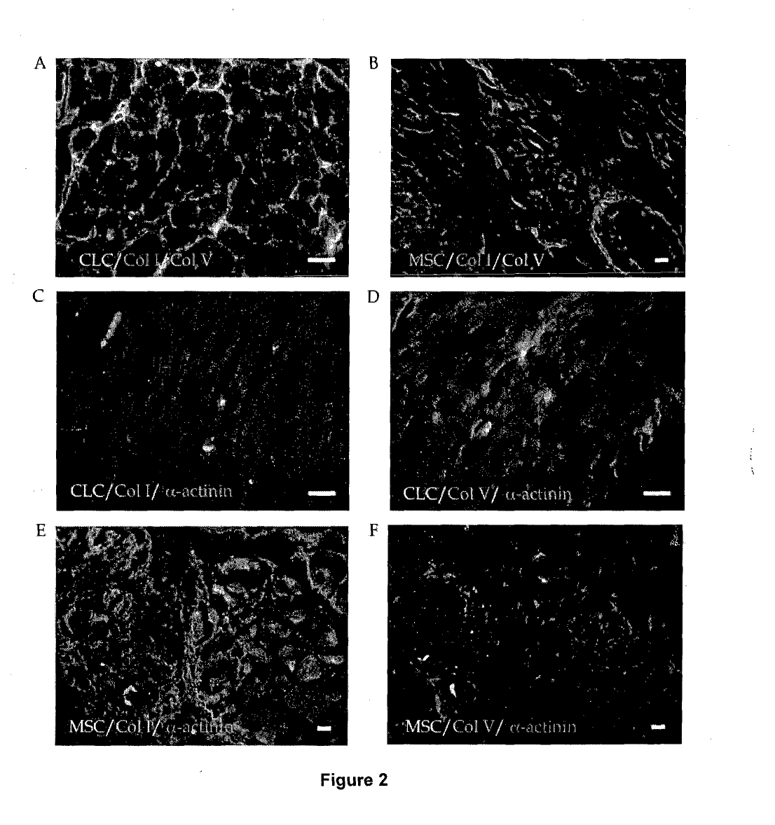

Myocardial transplanted CLCs were closely associated with collagen V matrix in the endomysial space in the peri-infarct border of the myocardium (FIG. 2A). In contrast, similarly transplanted MSCs were only found in collagen I-rich infarct despite the presence of isolated, collagen V-expressing, myofibres at the infarct borders (FIG. 2B). Furthermore, CLCs were often intimately engrafted among α-actinin stained native cardiomyocytes that were surrounded by collagen V, but not collagen I, matrices (FIGS. 2C and 2D). On the contrary, transplanted MSCs were sequestered in the infarct that was dominated with collagen I matrix and isolated from viable and α-actinin stained myocardium that expressed collagen V matrix (FIG. 2E).

CLC Therapy at High Doses Improves Cardiac Hemodynamics

Consistent with their muscular engraftment, LV echocardiography confirmed a better cardiac performance of transplanted CLCs, 6 weeks post cell transplant (Table 4). Transplanted CLCs (2.2±0.3 mm, p<0.05), but not MSCs (2.1±0.3 mm), improved LV anterior wall thickness as compared to control infarcted animal (1.8±0.4 mm). Nevertheless, other cardiac parameters indicated that CLCs and MSCs contributed comparably to functional improvements by reducing chamber dilatation and moderating negative LV remodeling.

TABLE 4
Ultrasound echocardiography assessment of post cellular therapy
treated rats. 2D ultrasound echocardiography assessments showed
significant improvements in cell transplanted animals.

| | SF | MSC | CLC | Statistical |
|---|---|---|---|---|
| M-Mode | (n = 15) | (n = 19) | (n = 17) | Significance |
| LVIDed (mm) | 7.7 ± 0.9 | 7.3 ± 0.6 | 7.1 ± 0.7 | NS |
| LVIDes (mm) | 5.0 ± 1.1 | 4.2 ± 0.6⁺ | 4.1 ± 0.9* | ⁺p < 0.05 vs. SF |
| | | | | *p < 0.01 vs. SF |
| IVSed (mm) | 1.3 ± 0.2 | 1.3 ± 0.1 | 1.4 ± 0.1 | NS |
| IVSes (mm) | 1.8 ± 0.4 | 2.1 ± 0.3 | 2.2 ± 0.3* | *p < 0.05 vs. SF |
| AWT (%) | 49.8 ± 16.3 | 53.6 ± 14.7⁺ | 54.0 ± 15.6* | ⁺p < 0.05 vs. SF |
| | | | | *p < 0.05 vs. SF |
| FS (%) | 35.0 ± 5.6 | 42.4 ± 5.3⁺ | 43.1 ± 6.6* | ⁺p < 0.05 vs. SF |
| | | | | *p < 0.005 vs. SF |
| EF (%) | 60.4 ± 7.9 | 68.7 ± 6.3⁺ | 69.8 ± 9.5* | ⁺p < 0.05 vs. SF |
| | | | | *p < 0.005 vs. SF |

SF: Serum free control; CLC: Cardiomyocyte-like-cells; MSCs: Mesenchymal stem cells; LVIDed: Left ventricular internal dimension at end diastolic; LVID: Left ventricular internal dimension at end systolic; IVSed: Interventricular septum at end diastolic, IVSes: Interventricular septum at end systolic; AWT: Anterior wall thickening; FS: Fractional shortening; EF: Ejection fraction.

Discussion

Integrins and ECM are important modulators of stem cell behaviours. To date, cardiac cell therapy supported only modest benefits, likely due to low engraftment of transplanted cells in the infarcted myocardium. Exploration of specific integrin/ECM interaction may improve engraftment and survival of transplanted cells and ultimately, mechanical function of the heart. The current study examines integrin/ECM interactions on cardiac gene expression of CLCs and distribution of transplanted CLCs in infarcted myocardium.

The distribution and quantity of type I and III collagens in the heart play an important role in maintaining cardiac function. Alterations of collagen population and distribution in the myocardium affect size and shape of the heart chambers as well as myocardial diastolic and systolic function (Cleutjens et al., 1995a; Janicki & Brower, 2002). However, it is unclear if such alterations could affect stem cell migration and differentiation in the myocardium.

A previous report demonstrated that CLCs showed preferential adhesion to collagen V over collagen I matrix by interacting with subsets of integrins (Shim et al., 2004; Tan et al., 2010). van Laake et al. (2010) reported that pre and post transplanted human embryonic cardiomyocytes (hESC-CM) express integrins matching ECM types they encountered in their environment. Therefore, the integrin modulating role of collagen V may aid in the observed retention of the myocardial transplanted CLCs. Furthermore, intimate engraftment of the transplanted CLCs with collagen V-expressing, α-actinin positive, native cardiomyocytes supports an unique role of collagen V in the myocardium. Moreover, differential expression of α₁ and β₃ integrin between collagen I and V cultured CLCs coupled with the preferential homing demonstrated between transplanted MSCs and CLCs suggested a key role of collagen V matrix, not only in cellular retention, but cardiac differentiation of the transplanted stem cells. This is consistent with modulation of cardiac gene expression of CLCs demonstrated in relation to α₁ and αᵥβ₃ neutralisation in vitro, although such relationship was not examined in vivo. Nevertheless, the comparable cardiac outcomes achieved in spite of selective homing of the transplanted cells, indicates that different reparative mechanisms may be initiated by MSCs and CLCs. Despite a positive trend of systolic improvement by CLCs, further mechanistic studies are warranted to discern their specific contribution to systolic and diastolic components of cardiac performance.

Integrin α₁ is known to transduce ECM signals to the cytoskeleton that activate downstream mitogen activated protein kinase (MAPK) and extracellular signal-regulated kinase 1 (ERK1) signalling pathways that phosphorylate and activate GATA4 (Akazawa & Komuro, 2003). However, GATA4 expression was unaffected by integrin α₁ neutralisation despite the upregulated Trop C and Trop T belonging to downstream genes known to be activated by GATA4 (Liang et al., 2001; Tidyman et al., 2003). Similarly, neutralisation of αᵥβ₃ integrin attenuated Trop C expression despite GATA4 was previously shown to be unaffected by neutralisation of αᵥβ₃ (Tan et al., 2010). It is unclear if the modulation of myofilamental gene expression demonstrated was secondary to other nuclear transcription factor. However, integrins are known to mechanotransduce signals to activate Raf-MEK-ERK-1/2 cascade that has been shown to elicit cardiomyocyte growth, increased fetal-gene expression and cytoskeletal reorganization in neonatal cardiomyocytes (Lorenz et al., 2009). Nevertheless, it is unclear if reduced expression of integrin demonstrated on either collagen surface as compared to CLCs cultured on uncoated polystrene surface was associated with enhanced proliferation of CLCs as previously reported (Tan et al 2010). However, contrary to data from Tan et al 2010, SKAA was not down regulated by integrin αᵥβ₃ neutralisation in the current study. This could be due to donor variations. Indeed, donor variation in integrin expression has been documented from different bone marrow isolates and passage numbers, resulting in different growth and proliferation potential (ter Brugge et al., 2002).

Despite beneficial effect of collagen V on cardiac gene expression and stem cell distribution, it should be noted that collagen distribution in the infarcted rat hearts may be different from humans during MI. Furthermore, a 3D structure like the heart may transmit different environmental cues to integrins as compared to 2D environments provided in tissue culture experiments. It remains to be determined whether inhibitory antibodies may transactivate other integrin receptors during epitope occupancy. In addition, the promiscuity of integrins renders it technically challenging to identify whether a single integrin or interplay of synergistic interactions between a few integrins is required for regulation of cardiac gene expression. Future studies employing siRNA techniques that selectively silence α₁ or αᵥβ₃ integrin may provide additional information regarding the regulation of cardiac gene expression of CLCs on collagen V matrix ex vivo or in the transplanted milieu of infarcted myocardium.

In conclusion, this study indicates that α₁ and αᵥβ₃ integrins drive cardiac gene expression of CLCs. Integrin families and ECM are important regulators of cardiac differentiation and myocardial distribution of adult MSCs and CLCs. Specific modulation of interaction between subclasses of collagen and integrin subunits in the post-infarct myocardial ECM could potentially offer a unique opportunity in cardiac regenerative medicine.

### REFERENCES

Akazawa, H., & Komuro, I. (2003). Roles of cardiac transcription factors in cardiac hypertrophy. Circ Res, 92(10), 1079-1088.
Boudoulas, K. D., & Hatzopoulos, A. K. (2009). Cardiac repair and regeneration: The rubik's cube of cell therapy for heart disease. Dis Model Mech, 2(7-8), 344-358.
Brancaccio, M., Hirsch, E., Notte, A., Selvetella, G., Lembo, G., & Tarone, G. (2006). Integrin signalling: The tug-of-war in heart hypertrophy. Cardiovasc Res, 70(3), 422-433.
Breuls, R. G., Klumpers, D. D., Everts, V., & Smit, T. H. (2009). Collagen type v modulates fibroblast behavior dependent on substrate stiffness. Biochem Biophys Res Commun, 380(2), 425-429.
Chacko, S. M., Khan, M., Kuppusamy, M. L., Pandian, R. P., Varadharaj, S., Selvendiran, K., et al. (2009). Myocardial oxygenation and functional recovery in infarct rat hearts transplanted with mesenchymal stem cells. Am J Physiol Heart Circ Physiol, 296(5), H1263-1273.
Cleutjens, J. P., Kandala, J. C., Guarda, E., Guntaka, R. V., & Weber, K. T. (1995b). Regulation of collagen degradation in the rat myocardium after infarction. J Mol Cell Cardiol, 27(6), 1281-1292.
Cleutjens, J. P., Verluyten, M. J., Smiths, J. F., & Daemen, M. J. (1995a). Collagen remodeling after myocardial infarction in the rat heart. Am J Pathol, 147(2), 325-338.
Djouad, F., Delorme, B., Maurice, M., Bony, C., Apparailly, F., Louis-Plence, P., et al. (2007). Microenvironmental changes during differentiation of mesenchymal stem cells towards chondrocytes. Arthritis Res Ther, 9(2), R33.
Feygin, J., Mansoor, A., Eckman, P., Swingen, C., & Zhang, J. (2007). Functional and bioenergetic modulations in the infarct border zone following autologous mesenchymal stem cell transplantation. Am J Physiol Heart Circ Physiol, 293(3), H1772-1780.
Fichard, A., Kleman, J. P., & Ruggiero, F. (1995). Another look at collagen v and xi molecules. Matrix Biol, 14(7), 515-531.
Gaudette, G. R., & Cohen, I. S. (2006). Cardiac regeneration: Materials can improve the passive properties of myocardium, but cell therapy must do more. Circulation, 114(24), 2575-2577.
Gronthos, S., Simmons, P. J., Graves, S. E., & Robey, P. G. (2001). Integrin-mediated interactions between human bone marrow stromal precursor cells and the extracellular matrix. Bone, 28(2), 174-181.
Hannigan, G. E., Coles, J. G., & Dedhar, S. (2007). Integrin-linked kinase at the heart of cardiac contractility, repair, and disease. Circ Res, 100(10), 1408-1414.
Janicki, J. S., & Brower, G. L. (2002). The role of myocardial fibrillar collagen in ventricular remodeling and function. J Card Fail, 8(6 Suppl), S319-325.
Liang, Q., De Windt, L. J., Witt, S. A., Kimball, T. R., Markham, B. E., & Molkentin, J. D. (2001). The transcription factors gata4 and gata6 regulate cardiomyocyte hypertrophy in vitro and in vivo. J Biol Chem, 276(32), 30245-30253.
Linehan, K. A., Seymour, A. M., & Williams, P. E. (2001). Semiquantitative analysis of collagen types in the hypertrophied left ventricle. J Anat, 198(Pt 1), 83-92.
Lorenz, K., Schmitt, J. P., Vidal, M., & Lohse, M. J. (2009). Cardiac hypertrophy: Targeting raf/mek/erk1/2-signaling. Int J Biochem Cell Biol, 41(12), 2351-2355.
Lu, L., Zhang, J. Q., Ramires, F. J., & Sun, Y. (2004). Molecular and cellular events at the site of myocardial infarction: From the perspective of rebuilding myocardial tissue. Biochem Biophys Res Commun, 320(3), 907-913.
Michel, J. B. (2003). Anoikis in the cardiovascular system: Known and unknown extracellular mediators. Arterioscler Thromb Vasc Biol, 23(12), 2146-2154.
Niyibizi, C., Chan, R., Wu, J. J., & Eyre, D. (1994). A 92 kda gelatinase (mmp-9) cleavage site in native type v collagen. Biochem Biophys Res Commun, 202(1), 328-333.
O'Kane, S., & Ferguson, M. W. (1997). Transforming growth factor beta s and wound healing. Int J Biochem Cell Biol, 29(1), 63-78.
Perkins, A. D., Ellis, S. J., Asghari, P., Shamsian, A., Moore, E. D., & Tanentzapf, G. (2010). lntegrin-mediated adhesion maintains sarcomeric integrity. Dev Biol, 338(1), 15-27.
Ross, R. S., & Borg, T. K. (2001). Integrins and the myocardium. Circ Res, 88(11), 1112-1119.
Ruggiero, F., Champliaud, M. F., Garrone, R., & Aumailley, M. (1994). Interactions between cells and collagen v molecules or single chains involve distinct mechanisms. Exp Cell Res, 210(2), 215-223.
Shim, W. S., Jiang, S., Wong, P., Tan, J., Chua, Y. L., Tan, Y. S., et al. (2004). Ex vivo differentiation of human adult bone marrow stem cells into cardiomyocyte-like cells. Biochem Biophys Res Commun, 324(2), 481-488.
Spinale, F. G. (2007). Myocardial matrix remodeling and the matrix metalloproteinases: Influence on cardiac form and function. Physiol Rev, 87(4), 1285-1342.
Sun, Y., & Weber, K. T. (2000). Infarct scar: A dynamic tissue. Cardiovasc Res, 46(2), 250-256.
Tan, G., Shim, W., Gu, Y., Qian, L., Chung, Y. Y., Lim, S. Y., et al. (2010). Differential effect of myocardial matrix and integrins on cardiac differentiation of human mesenchymal stem cells. Differentiation, 79(4-5), 260-271.
ter Brugge, P. J., Torensma, R., De Ruijter, J. E., Figdor, C. G., & Jansen, J. A. (2002). Modulation of integrin expression on rat bone marrow cells by substrates with different surface characteristics. Tissue Eng, 8(4), 615-626.
Tidyman, W. E., Sehnert, A. J., Huq, A., Agard, J., Deegan, F., Stainier, D. Y., et al. (2003). In vivo regulation of the chicken cardiac troponin t gene promoter in zebrafish embryos. Dev Dyn, 227(4), 484-496.
van Laake, L. W., van Donselaar, E. G., Monshouwer-Kloots, J., Schreurs, C., Passier, R., Humbel, B. M., et al. (2010). Extracellular matrix formation after transplantation of human embryonic stem cell-derived cardiomyocytes. Cell Mol Life Sci, 67(2), 277-290.
Xu, X., Xu, Z., Xu, Y., & Cui, G. (2005). Effects of mesenchymal stem cell transplantation on extracellular matrix after myocardial infarction in rats. Coron Artery Dis, 16(4), 245-255.

1. A method for preparing improved cardiomyocyte-like cells comprising the steps of:
(i) differentiating at least one multipotent or pluripotent cell into cardiomyocyte-like cell(s); and
(ii) contacting the cardiomyocyte-like cell(s) with at least one agent to promote and/or induce integrin subunit alpha-V (integrin V) activity, thereby modulating expression of at least one cardiac gene of said cardiomyocyte-like cells.

2. The method according to claim 1, wherein the at least one agent promotes and/or induces integrin subunit alpha-V activity by itself or as part of a dimer.

3. The method according to claim 1, wherein the at least one agent comprises an activating antibody, a RGD peptide and/or a constitutively active form of alpha-V integrin subunit.

4. The method according to claim 1, wherein promoting and/or inducing the activity of integrin subunit alpha-V comprises promoting and/or inducing integrin subunit alpha-V gene expression.

5. The method according to claim 4, wherein promoting and/or inducing the activity of integrin subunit alpha-V comprises promoting and/or increasing integrin subunit alpha-V gene expression at the transcriptional and/or translational levels.

6. The method according to claim 1, further comprising contacting the multipotent, pluripotent and/or cardiomyocyte-like cell(s) with collagen prior to or concomitant with the at least one agent.

7. The method according to claim 6, wherein the collagen comprises collagen IV, V and/or XI.

8. (canceled)

9. The method according to claim 1, wherein step (ii) comprises modulating cardiac gene expression at the transcriptional and/or translational levels.

10. The method according to claim 1, wherein step (ii) comprises enhancing cardiac gene expression.

11. The method according to claim 1, wherein the at least one cardiac gene comprises sarcomeric α-actin, sarcomeric α-actinin, desmin, skeletal/cardiac specific titin, sarcomeric α-tropomyosin, cardiac troponin I, sarcomeric MHC, SERCA2 ATPase, connexin-43, GATA binding protein 4 (GATA4), Nkx2.5, myocyte enhancing factor 2A, myocyte enhancing factor 2C, myocyte enhancing factor 2D, cardiac α-actin, skeletal muscle α-actin, cardiac troponin T, cardiac troponin C, and L-type calcium α1c.

12. (canceled)

13. (canceled)

14. (canceled)

15. The method according to claim 1, wherein the cardiomyocyte-like cells are suitable and/or for use in transplantation.

16. A method for screening for an agent capable of improving cardiomyocytes differentiated from at least one multipotent or pluripotent cell comprising screening for a candidate agent capable of promoting and/or inducing integrin subunit alpha-V activity.

17. The method according to claim 16, comprising the steps of:
(i) providing cardiomyocyte-like cell(s) differentiated from at least one multipotent or pluripotent cell;
(ii) contacting the cardiomyocyte-like cell(s) with the candidate agent; and
(iii) (a) screening for a candidate agent capable of modulating the expression of at least one cardiac gene when compared to the expression of said cardiac gene in cardiomyocyte-like cells absent the candidate agent; and/or
(b) determining if the candidate agent promotes and/or induces integrin subunit alpha-V activity.

18. The method according to claim 17, wherein the ability of the candidate agent to modulate the expression of the at least one cardiac gene and/or to promote and/or induce integrin subunit alpha-V activity is indicative of its ability to improve cardiomyocytes differentiated from at least one multipotent or pluripotent cell.

19. (canceled)

20. The method according to claim 16, comprising detecting for enhanced expression of GATA4, Nkx2.5, cardiac α-actin, cardiac troponin T, and/or cardiac troponin C genes in cardiomyocyte-like cells differentiated from at least one multipotent or pluripotent cell contacted with the candidate agent.

21. (canceled)

22. (canceled)

23. The method according to claim 16, comprising detecting and comparing the expression of at least one cardiac gene in cardiomyocyte-like cells contacted with and absent the candidate agent.

24. The method according to claim 17, wherein modulating the expression of at least one cardiac gene is at the transcription and/or translation level.

25. The method according to claim 24, comprising detecting for the transcription and/or translation product.

26. The method according to claim 25, wherein detection comprises using real-time PCR, microarray analysis, ELISA and/or immunoblotting.

27. The method according to claim 1, wherein the method is an in vitro method.

28. The method according to claim 16, wherein the method is an in vitro method.
